(19) United States
(12) Patent Application Publication
(10) Pub. No.: US 2013/0121982 A1
(43) Pub. Date: May 16, 2013

(54) ANTIDOTE TO THE BITE OF VENOMOUS SNAKES

(71) Applicant: David Satanassi, Mercato Saraceno (IT)

(72) Inventor: David Satanassi, Mercato Saraceno (IT)

(21) Appl. No.: 13/811,066

(22) PCT Filed: Jul. 21, 2011
(86) PCT No.: PCT/IB2011/053256

§ 371 Date: Jan. 28, 2013

(30) Foreign Application Priority Data
Jul. 22, 2010 (IT) MI2010A001348

(51) Int. Cl. A61K 38/54

(52) U.S. Cl. CPC A61K 38/54
USPC 424/94.2

(57) ABSTRACT

The present invention concerns a composition comprising a low dosage of at least one venom derived from a venomous snake, characterised in that said composition is diluted in an aqueous medium according to the Sequential Kinetic Activation method. In addition, the present invention concerns the use of said composition as an antidote against the poisoning caused by the bite of a venomous snake.

The present invention concerns a composition comprising a low dosage (homoeopathic dose) of snake venom and its use as an antidote to the bites of venomous snakes.

The ophidians, also known as venomous snakes, are classified into three families: the Colubrids, the Elaphids and the Viperids.

The family of the Viperids is distinguished into two sub-families: the Viperids strictly speaking and the Crotalids. All the Viperids are venomous.

Venom evolved to allow “extracorporeal digestion”. In fact, it is typical of the ophidians to swallow their prey whole without lacerations or mastication. The need to do this caused the transformation of the secretions of the salivary glands into a substance with proteolytic-enzymatic action, to the point where a lethal level is reached, both in relation to the quantity produced, and in relation to its concentration.

A snake's venom contains a series of toxic substances with different functions. In particular, these toxic substances can be enzymes with local action, or enzymes with systemic, neurotoxic, vasoactive or haemoactive action.

Toxins with “local action” can be distinguished into “myonecrotic” toxins and toxins with “systemic action” (or “myoglobinuric toxins”) and are phospholipases which trigger violent and sometimes unbearable pain at the site where the venom is injected.

The neurotoxic action develops with α-neurotoxins present in the venom of the majority of Elaphids (cobra, mamba, coral snake and Australian sea-snakes). They act by blocking the interaction of acetylcholine with the nicotinic receptor by creating a phenomenon of post-synaptic inhibition.

The neurotoxins which however act in the pre-synaptic alter the release of acetylcholine from the motor terminations, producing two opposite phenomena: inhibition of release of the mediator, or facilitation of its release. The overall effect is, however, identical and occurs with the blocking of neuromuscular transmission.

β-neurotoxins are phospholipasic enzymes of type A2 and are the most toxic component of ophidians' venom.

Toxins which act on the cardiovascular system also have a marked tropism for the heart, and for this reason they are defined as “cardiotoxins” and act directly on the cardiac fibres, depolarising them.

The principal enzymes comprised in the venom of a snake are: cholinesterase, fibrinogen-coagulase/thrombin, phosphatase, phostatidase, hyaluronidase, haemorrhagic factors, accelerating factors for blood coagulation, inhibiting factors for blood coagulation, phospholipase, L-amino acid-oxidase, proteolytic, vasoactive and/or anticoagulant enzymes. Some of these enzymes are common to all snakes while others are typical of a specific kind.

The venom is inoculated at the moment of the bite through the teeth which, in the solenoglyphic ophidians, are hollow like syringes, to allow effective inoculation in depth into the tissues of the prey.

Vipers can inject with a single bite from 5 to 25 mg on average of venom (in terms of the weight of dry venom). For example, the lethal dose for a dog of average build, weighing around 20 kg, is about 3.5 mg. In these conditions 60-70% of dogs that have been bitten die.

Following a snake-bite, alterations to creatine-kinase levels can be observed. Values of this enzyme can triple compared with the maximum threshold, indicating a state of muscular pain induced by the bite and the consequent lethargy induced by locomotor damage.

Massive myoglobinuria can be observed which appears in the hours succeeding the bite and is indicative and pathognomonic for the bite itself. Massive myoglobinuria, combined with the anamnesis, could lead the doctor to direct his/her suspicions to presumed poisoning by ophidian bite, even if there have not been direct observations of the event.

The weight of the individual who has been bitten, their state of health and the location of the puncture strongly impact on the lethality due to the bite of a venomous snake.

One of the possible causes of the increase in deaths following a bite by venomous ophidians, particularly deaths of animals, is attributable to the reactivity of the subjects. In fact, what occurs following the bite of a venomous snake is, often, a strong allergic response which can lead to death.

The remedy historically utilised for ophidian bites is the use of hyperimmune serums.

Hyperimmune serum is obtained by inoculation of the venom of a snake, for example a viper, into healthy horses which have been prepared for this kind of therapy.

After a suitable period of immune response, the whole blood of the horse is taken to obtain the serum.

IgGs are inoculated subcutaneously or directly intravenously for the purpose of opposing the effects of the venom by inactivating it or, more accurately, chemically binding to the polypeptide chains constituting the venom, and thus enabling them to be recognised and caught by the macrophages which inactivate them by digesting the immunocomplexes.

Antiserums are available as purified tetravalent antiophidian serum of equine origin, or as fragments of F(ab) antibodies of bovine origin (the latter being too expensive to be routinely used).

Purified tetravalent antiophidian serum, acting against the four European vipers, is administered subcutaneously when it is possible to trace the bite, or intramuscularly.

Its efficacy is greater the earlier it is administered. The therapeutic action of the serum is expressed in the course of a period ranging from a few minutes to several hours, depending on the time which elapses between the accidental event and first aid treatment.

The serum is, however, not recommended for cardiopathic patients or those allergic to horse and/or bovine proteins; or for patients with previous episodes of asthmatic illness or allergies (currently around 25% of the population).

The difficulties and limits of use of hyperimmune serum as a solution to the poisoning produced by ophidian bites lie in the following areas: 1) the difficulty of procuring the serum and 2) the collateral effect of anaphylactic shock following its administration. This shock can lead to death and can therefore be as dangerous as the snakebite itself. The serum must in fact always be administered by skilled personnel in a facility equipped to deal with the possibility of a probable anaphylactic shock occurring.

As an alternative to hyperimmune serum it is possible to apply to the site of the bite an electrostatic device capable of neutralising the venom through an electrophoretic process.

However, the efficacy of this method has not yet been scientifically proved; furthermore, its application is limited by the fact that it must be possible to identify the area of the bite and therefore the point of inoculation of the venom, which is not easy to do.

The technical problem at the base of the present invention concerns the identification of a remedy for the poisoning due to bites by venomous snakes which overcomes the limits of the known art discussed above.

There exists in the sector a need to make available a method which, unlike the methods currently available, is easy to procure and execute and is safe, i.e. free from dangerous collateral effects such as the occurrence of anaphylactic shock.

The present invention resolves the technical problem stated above with a composition comprising at least one venom derived from a venomous snake, characterised in that said composition is diluted in an aqueous medium according to the Sequential Kinetic Activation method.

In addition, the present invention concerns the use of said composition as an antidote against the poisoning caused by the bite of a venomous snake. The antidote can be utilised both for veterinary and for human use.

In a preferred embodiment of the invention the composition comprises at least one venom extracted from the veneniferous gland of a snake. Preferably the at least one venom is a mixture of 2-10 venoms extracted from various venomous snakes, more preferably it is a mixture of 3-6 venoms.

More preferably, the at least one venom comprises the venom of the ophidian which inflicted the bite.

The at least one venom is extracted from at least one snake selected from among Vipera aspis (Asp Viper), Lachesis mutus, Naja tripudians, Timber rattlesnake (Crotalus horridus), Vipera ammodytes, Vipera russel, Agkistrodon anctarticus, Bungarus fasciatus, Botrops lanceolatus, Bitis gabonica, Dendroaspis sp, Elaps corallines, Bushmaster (Surukuku or Trigonocephalus lachesis) and Asian king cobra (Naja tripudians).

Preferably, the at least one venom is extracted from at least one snake selected from among Vipera aspis (Asp Viper), Timber rattlesnake (Crotalus horridus), Bushmaster (Surukuku or Trigonocephalus lachesis) and Asian king cobra (Naja tripudians).

In an even more preferred form of the invention, the at least one venom is the venom extracted from the Vipera Aspis.

In another embodiment, the at least one venom is a mixture of the venoms extracted from Vipera aspis (Asp Viper), Timber rattlesnake (Crotalus horridus), Bushmaster (Surukuku or Trigonocephalus lachesis) and Asian king cobra.

Said extracted venom is diluted in an aqueous solution, preferably an isotonic solution, more preferably a physiological solution.

Said dilution is performed using the technique of “Sequential Kinetic Activation” (S.K.A.). This dilution protocol is known in the sector and consists of a series of dilutions and “activations” (for example by means of agitation/succussion) of the active principle until arriving at the so-called “low dose” or homoeopathic dose.

In the context of the present invention the protocol preferably used to dilute the venom is of Hahnemann type. This protocol provides for the repeated dilution of a substance, in this case the venom, in a solvent which, preferably, is water or a physiological solution.

The venom can be diluted at each stage of dilution by about a hundred times.

After each dilution the sample is agitated about a hundred times. In particular, the agitation, in the context of Hahnemann type dilutions, takes the name of succussion.

After each succussion stage, about a hundredth part of each diluted sample is further diluted about a hundred times. The dilution is followed by succussion as first explained. Dilution and succussion stages can be repeated a variable number of times. For example a dilution-succussion performed (according to protocol S.K.A.) 9 times is defined as 9 centesimal Hahnemann (9 CH), a dilution-succussion performed 30 times is defined as 30 centesimal Hahnemann (30 CH) and a dilution-succussion performed 200 times is defined as 200 centesimal Hahnemann (200 CH).

Using a Hahnemann type dilution protocol it is observed that there are still molecules present of the original venom in the various stages of dilution-succussion. In particular, this has been found for dilutions from the ninth centesimal Hahnemann to the twelfth dilution.

For example, the residual quantity of original venom in a 9 CH dilution is 0.000000000000000001 grams of venom in one ml of composition.

According to a particular aspect of the invention, equal parts can be mixed of at least one Hahnemann 9 CH, 30 CH or 200 CH dilution of the following venoms: Lachesis M., Crotalus H., Naja Trip., Vipera Aspis.

For example, 0.25 ml can be mixed of each of said venoms. In one embodiment of the invention the composition is administered by the oral, intramuscular or intraperitoneal routes. Preferably said composition is administered subcutaneously.

The points of subcutaneous administration are preferably the injection points of the venom, i.e. the site where the snakebite is located.

The administration posology comprises the inoculation of one or more individual doses of composition of the invention. Preferably, the administration comprises the inoculation of 3-4 consecutive doses of homoeopathic composition, at intervals of 3-10 minutes from each other.

In another embodiment, the administration comprises the inoculation of 2-3 doses of homoeopathic composition per day for the 3-7 days following the bite.

### EXAMPLE 1

With the object of covering the most possible symptoms derived from poisoning by snakebite, the composition of the invention was prepared by mixing the venoms from the following snakes: Vipera Aspis (Asp Viper), Timber rattlesnake (Crotalus horridus), Bushmaster (Surukuku or Trigonocephalus lachesis) and Asian king cobra (Naja tripudians).

The venom was obtained by extracting the snake's veneniferous glands into special containers, called “milkers”.

The venoms were mixed together only after a dilution/succussion protocol was performed for each of them (protocol S.K.A. Sequential Kinetic Activation).

In particular, each venom was diluted 200 CH. The composition was produced in isotonic solution according to the Hahnemann protocol explained above. After dilution the four compositions were mixed to obtain the composition to be administered. The choice of the route for administration of the composition of the present invention depends very much on the patient to be treated. While adjustments were being made to the method, the composition was administered to animals, in particular to dogs and cats: specifically, 137 dogs and 3 cats.

The composition was administered subcutaneously.

The dose of one millilitre of isotonic solution of the composition of the invention was administered subcutaneously at two points: the first at the site of inoculation of the snake's venom; the second dose (simultaneously) subcutaneously in the thoracic region.

Administration of a third dose as well is recommended ten minutes after the previous doses, if there have not been clinical signs indicating partial remission of the symptoms, or an aggravation of said symptoms (cardiocirculatory collapse, hypothermia, pale and cyanotic mucosas).

On the doctor's advice subsequent doses of the composition were given until the symptoms diminished.

In particular, in the animals which underwent snakebite, two administrations daily were given every 12 hours, for the seven days following the bite.

After the first injection it was necessary to reach the nearest veterinary centre and continue the support treatment with alkalizing rehydratants (Ringer lactate) until the patient regained spontaneous urination and a return to normal body temperature was observed.

The composition administered gave very obvious results in the recovery of functions and the remission of symptoms in times varying from 30 minutes to two weeks in the most serious cases.

### EXAMPLE 2

The composition which is the subject of the invention was administered subcutaneously to 141 dogs and 3 cats.

In particular, in 73% of the subjects there was the certainty of a snakebite, whereas in 27% of the cases clinical signs attributable to a bite by the ophidian were observed.

During the clinical examination the following parameters were observed: cardiocirculatory collapse (84% of cases presented this symptom), shock (60% presented this symptom), exhaustion and prostration (98% presented this symptom), fall in blood pressure, weak and rapid pulse (98% presented this symptom) or bradypnoea.

Eight animals were treated with the method of the invention after the classical antishock treatments (based on phleboclysis and cortisones at antishock dosages) did not effect any improvement, indeed the conditions seriously worsened.

Three of these subjects were treated with the composition of the invention more than 48 hours after the bite.

Notwithstanding that they had been treated with fluidotherapy and cortisone in cases of shock, (0.5/20 mg/kg 2/3 times/day) the animals went into very serious conditions connected with haematological disorders such as an increase in bilirubinaemia, granulocytosis, anaemia, increase in azotaemia, creatininaemia, GOT, GPT and CK. These conditions were obviously the consequence of the action of the venom.

Furthermore, the haematocrit value was below 20%, which necessitated the transfusion of whole blood from a compatible donor, to restore blood volume and consequently lower the metabolic acidosis which arose through the low inflow of oxygen.

Four subjects died.

One subject died 72 hours after the bite.

This subject had not received any specific examination. It died without having had (at its home site) any attention during the first two hours.

The second subject died through the voluntary intervention of its owner, who, 72 hours after the snakebite, requested that it be put down.

The third and unique case died in the clinic after a sharp reaction to the emergency protocol. In particular, the animal died four hours after first aid and five hours after the snakebite.

The fourth animal treated showed a good response at the acute stage, but the home treatment was interrupted through lack of interest by its owners, so the subject died.

In conclusion, 117 dogs and 3 cats, brought for urgent examination following poisoning by snakebite, showed complete and lasting remission of the symptoms following administration of the composition which is the subject of the present invention.

The composition of the invention therefore demonstrates the efficacy of an antidote. In particular, it shows itself to be effective at twelve hours after the bite and up to two weeks.

By contrast, the subject treated with anti-ophidic serum at the recommended doses and with the procedures indicated twelve hours after the bite died without remission of the symptoms.

The dogs brought for urgent examination showed the following general acute symptoms: cardiocirculatory collapse: (sharp fall in systemic pressure, tachycardia, faint, rapid and frequent pulse, bradypnoea, 92% of subjects); fall in body temperature, below 38.5° C., with low points of 35.5° C.; difficulty in walking, locomotor ataxia, catatonia, loss of strength, initially in the hind quarters and later in all the skeletal muscles, as an obvious effect of the cardiocirculatory collapse or because of the intense presumed pain at the inoculation site; gastroenteric symptoms, sialorrhea, the more intense the closer the bite was to the facial and oral-labial region, vomiting in 20% of cases, diarrhoeic discharge irrespective of the quality and quantity of food ingested; neurological symptoms: palpebral ptosis and lagophthalmia, amaurosis, anisocoria, nystagmus, mydriasis in case of temporary blindness (up to 72 hours 2%), delayed blink reflex, convulsive crises, stuporous coma, excitable phases alternating with comatose states, lingual ptosis, Horner-like syndromes, delays in proprioceptive and nociceptive postural reflexes, 18% of subjects treated.

Urinary symptoms concern the tendency to stagnation and oliguria (64%) in the first twelve hours up to urinary blockage.

Local symptoms regard local oedema at the inoculation site (60%), tumefaction, reddening with a tone that becomes purple over a period of hours with suffusions characteristic of the action of the venom on the skin and in the derma; the oedema can last as much as 120 hours or appear at a later stage or show other sites of inoculation with the venom when there is more than one point; in 34% of the subjects bitten, bacterial infection at the inoculation site with production of exudate of microbial origin with secondary irruption because of the proteolysis caused by the enzymes on the tegumentary layers.

As regards symptoms in the chronic course where death does not supervene and/or where conventional treatment has been applied, they are as follows: myoglobinuria, haematuria, haemolytic and non-haemolytic anaemia, delays in coagulation because of exhaustion of so-called “consumption” coagulation factors, with hypocoagulability of the blood, very marked jaundice with pre- and post-hepatic bilirubin values of 500 and beyond, alteration of fibrinogen, anorexia, vomiting, hepatic insufficiency, metabolic acidosis, dyspnoea, haematocrit below 20, granulocytaemia higher than 15000, up to 30000, tachypnoea, “Kuhsmall” type breathing, renal insufficiency, surges in azotaemia and creatininaemia higher respectively than 70 and 4 mg/dl, primary and secondary cardiocirculatory insufficiency, both because of the direct action of the venom on the myocardial cells and on volaemic decompensation, anaemia, jaundice, widespread oedema, urinary blockage and hypoxia.

All the symptoms listed above were present in the animals treated 48 hours after the snakebite. These symptoms, treated with the composition of the invention, came within the vital parameters in times ranging from five days after the snakebite, up to and not exceeding two weeks after.

Complete recovery of the haematocrit, red blood cells and platelets required more than thirty days in relation to the natural haemopoeisis induced by the anaemia.

Two subjects of female sex, respectively a 5-year old Lagotto Romagnolo and a Golden retriever aged seven years, were bitten while pregnant.

The former was treated with the composition which is the subject of the invention at one month into gestation, the latter, on the other hand, at two weeks into gestation.

The treatment showed itself to be free from mutagenic and teratogenic effects for both animals.

In fact, the pregnancy was favourably resolved for both bitches, who gave birth to live puppies free from obvious abnormalities at birth. Furthermore, the puppies did not show any clinical signs of suffering, immaturity or underweight.

The echographic check proved favourable at a week before the expected delivery date and the birth took place naturally.

In particular, the puppies of the second bitch, which had been bitten in a foetal period most susceptible to teratogenic effects on organ development, showed no clinical signs, congenital defects or abnormalities. She gave birth naturally.

It seems obvious that cortisone treatment would have induced abortion or reabsorption or possibly secondary metritis.

The extraordinary observation to report is how the snake venom's distribution metabolism has a sort of relative toxicity because it is channelled into or towards drains considered “limiting” or borderline.

This metabolism, in the pregnant bitch, did not have the slightest effect on organs to which the tropism of the venom is linked.

No damage was observed to the liver or the kidneys, or alteration to the composition of the blood, or alterations to the albumen/globulin ratio or to the plasmatic proteins, such as would necessarily produce haematological changes and therefore foetal damage.

The composition for use as an antidote according to the present invention has shown itself capable of modulating the response to the venom and its catabolism in an “intelligent” way, preserving the organs and the blood composition from the toxicity and thus preventing foetal damage or abortion.

In fact, in the case of the first bitch, her huge response to being bitten by the viper, which occurred in July (among other things, with a snake of remarkable size and therefore presumably with a very toxic bite) was of a predominantly neurological nature, with extreme lethargy, ataxia, general weakness, temporary blindness and the most extreme prostration for 72 hours.

However, the classic hepatic and renal symptoms, the appearance of jaundice and myoglobinuria were almost or completely absent.

This tends to indicate a difference between an absolute toxicity (due to the total dose injected) and a relative toxicity (where, for a given dose of venom, the response is individualised by an antidote principle with its own capacity for modulation with respect to the “vis medicatrix naturae” (Nature's healing power), in other words an individualised response for bringing about lesser damaging effects).

In the case of the second pregnant female also there was no foetal damage.

The seven puppies did not display, either at birth or after 50 days, any alterations attributable to states of foetal distress or genetic abnormalities.

In this case too the damage from the viper's bite, promptly treated, left only visible marks of a local type, with local swelling and oedema, slight lethargy and an absence of the organic symptoms which are typically reported in the statistics.

72 hours after the bite, and the incautious suspension of the treatment by the trainer, the animals displayed a slight recrudescence of the symptoms until subsequent administrations of the composition finally put an end to the effects of the bite.

In the same way as in the previous case, either the venom created or the treatment deviated or “displaced” the absolute toxicity into symptoms of “relative” gravity, in respect of the pregnancy that was in progress.

The best results in terms of general recovery, cessation of the state of shock, renal filtration capacity, appetite and absence of neurological symptoms, which occurred in the cases where intervention with the antidotes had been made earliest, were clinically manifested right from the first hour after the incident.

The rapidity and timeliness of the treatment with the composition of the invention shows how the effects of the venom are less and almost nonexistent when the treatment is administered promptly.

From this it is clear that this therapeutic procedure has a particular place of its own as an emergency medicine for prompt use.

1. A composition comprising at least 2-10 venoms of venomous snake, characterized in that said venoms are diluted in an aqueous medium according to the Sequential Kinetic Activation method.

2. The composition according to claim 1, comprising a mixture of 3-6 venoms.

3. The composition according to claim 1, wherein said venoms are extracted from a venomous snake selected among Vipera aspis (Asp viper), Lachesis mutus, Naja tripudians, Timber rattlesnake (Crotalus horridus), Vipera ammodytes, Vipera russel, Agkistrodon anctarticus, Bungarus fasciatus, Botrops lanceolatus, Bitis gabonica, Dendroaspis sp, Elaps corallines, Bushmaster (Surukuku or Trigonocephalus lachesis) and Asian king cobra (Naja tripudians).

4. The composition according to claim 3, wherein said venomous snake is preferably selected among Vipera Aspis (Asp viper), Timber rattlesnake (Crotalus horridus), Bushmaster (Surukuku or Trigonocephalus lachesis) or Asian king cobra (Naja tripudians).

5. The composition according to claim 1, wherein at least one venom is diluted by means of the dilution and activation protocol known as “Sequential Kinetic Activation”.

6. The composition according to claim 5, wherein said protocol comprises serial of sequential steps of dilution of said venom in said aqueous medium and agitation (succussion) of said diluted solution.

7. The composition according to claim 6, wherein said sequential steps comprise a dilution from 9 to 200 times.

8. The composition according to claim 6, wherein said sequential steps comprise a dilution from 9, 30 or 200 times.

9. The composition according to any claim 1, wherein said venom is diluted in an aqueous solution.

10. The composition according to claim 9, wherein said aqueous solution is an isotonic solution.

11. (canceled)

12. An antidote comprising the composition according to claim 1.

13. Method for the treatment of a poisoning caused by the bite of a venomous snake comprising the step of administering to an individual in need thereof an effective amount of the antidote according to claim 12.

14. Method according to claim 13 wherein the bite is of Vipera Aspis.

15. The composition according to claim 9, wherein said aqueous solution is a physiological solution.

16. Method according to claim 13, wherein the composition is administered subcutaneously.

17. Method according to claim 13, wherein the composition is administered subcutaneously in the site where the snakebite is located.

18. Method according to claim 13, wherein the administration comprises 3-4 consecutive doses of homeopathic composition, at intervals of 3-10 minutes from each other.

19. Method according to claim 13, wherein the administration comprises 2-3 doses of homeopathic composition per day for the 3-7 days following the bite.
